(19) United States
(12) Patent Application Publication
(10) Pub. No.: US 2021/0177026 A1
(43) Pub. Date: Jun. 17, 2021

(54) ENZYME COMPOSITION FOR SUGAR METABOLIC REGULATION

(71) Applicants: Ji Hwan HWANG, Suwon-si, Gyeonggi-do (KR); UNIVERSITY INDUSTRY FOUNDATION, YONSEI UNIVERSITY, Seoul (KR)

(72) Inventors: Ji Hwan HWANG, Suwon-si, Gyeonggi-do (KR); Eun Jig LEE, Seoul (KR); Cheol Ryong KU, Seoul (KR)

(73) Assignees: Ji Hwan HWANG, Suwon-si, Gyeonggi-do (KR); UNIVERSITY INDUSTRY FOUNDATION, YONSEI UNIVERSITY, Seoul (KR)

(21) Appl. No.: 16/761,811

(22) PCT Filed: Oct. 11, 2018
(86) PCT No.: PCT/KR2018/011957

§ 371 Date: May 6, 2020

(30) Foreign Application Priority Data
Nov. 7, 2017 (KR) 10-2017-0147635

(51) Int. Cl. A23L 29/00

(52) U.S. Cl. CPC A23L 29/06; C12Y 204/01018; C12Y 302/01001; C12Y 302/01003; C12Y 101/03004; C12Y 111/01006; C12Y 302/01108; C12Y 302/01002

(57) ABSTRACT

Disclosed is an enzyme composition for regulating sugar metabolism which can regulate the absorption of glucose into the body by converting the carbohydrates in food to a form of sugar that is not absorbed in the stomach and the like before being decomposed in the small intestine into glucose by the activity of various enzymes such as maltase, sucrase, or lactase and the like and absorbed, wherein the enzyme composition includes: one or more enzymes selected from the group consisting of glucoamylase, sucrase and lactase; glucose oxidase; and transglucosidase.

### TECHNICAL FIELD

The present invention relates to an enzyme composition for regulating sugar metabolism, and more particularly, to an enzyme composition for regulating sugar metabolism, which includes one or more enzymes selected from the group consisting of glucoamylase, sucrase and lactase; glucose oxidase; and transglucosidase.

### BACKGROUND ART

Saccharides are important substances constituting the living organism and used as an energy source, and particularly, glucose is very important as a pivotal compound of carbohydrate metabolism in the human body.

While sugar is an essential compound necessary for the human body, excessive ingestion of glucose may cause obesity and a metabolic imbalance may lead to various diseases such as diabetes.

A representative example of diseases caused by the imbalance of sugar metabolism is hyperglycemia. Hyperglycemia refers to a condition in which a blood sugar level is abnormally high. Physiological hyperglycemia is a natural phenomenon that is temporarily triggered after eating, but an increase in blood sugar level outside the allowable range is an important problem that progresses or has a possibility of progression to diabetes. When hyperglycemia becomes chronic, it may affect other biological organs, causing serious diseases in the kidneys, nerves, cardiovascular system and retina, and thus it is necessary to regulate blood sugar by the regulation of sugar metabolism.

To regulate such sugar metabolism, technologies using plant extracts or the like (Korean Patent Nos. 10-1193730 and 10-1561600) are disclosed. However, in most cases, they have mechanisms affecting sugar mechanisms and factors associated with other physiological activities in the living body, side effects cannot be completely excluded.

For this reason, the inventors intended to use an enzyme involved in sugar metabolism to exclude such side effects. Since the method using an enzyme involved in sugar metabolism is not a method affecting certain factors in the living body, side effects can be prevented, and thus it was expected that, if sugar ingested from food could be effectively converted into a form that is not be absorbed in the body, sugar metabolism could be regulated very safely.

The inventors investigated the above-described method of more effectively regulating sugar metabolism using an enzyme, confirming that, when a combination of an enzyme capable of converting carbohydrates into glucose and glucose oxidase and transglucosidase is ingested with food or immediately before or after the ingestion of food, carbohydrates are effectively converted into a type of sugar that is not first absorbed in the stomach before being decomposed into glucose and absorbed in the small intestine due to the actions of various enzymes such as maltase, sucrase, lactase, etc., thereby preventing a rapid increase in blood sugar due to the absorption of glucose in the body. Therefore, the present invention was completed.

### DISCLOSURE

### Technical Problem

Therefore, the main object of the present invention is directed to providing an enzyme composition, which may have a low possibility of side effects since it does not affect other physiological activities in vivo other than the digestion of carbohydrates, and may effectively regulate sugar metabolism by effectively converting carbohydrates into a form of sugar that is not absorbed in the stomach before being decomposed into glucose due to the actions of several enzymes such as maltase, sucrase, lactase, etc. in the small intestine and absorbed in the body.

### PRIOR ART DOCUMENTS

Korean Patent No. 10-1193730

Korean Patent No. 10-1561600

### Technical Solution

In one aspect, the present invention provides an enzyme composition for regulating sugar metabolism, which includes one or more enzymes selected from the group consisting of glucoamylase, sucrase and lactase; glucose oxidase; and transglucosidase.

The enzyme composition of the present invention may further include catalase, in addition to the enzymes.

The enzyme composition of the present invention may include all of glucoamylase, sucrase, lactase, glucose oxidase, transglucosidase and catalase.

The enzyme composition of the present invention may further include one or more types of amylases selected from α-amylase and β-amylase, in addition to the above enzymes.

### Advantageous Effects

An enzyme composition for regulating sugar metabolism of the present invention can regulate the absorption of glucose in the body by converting carbohydrates in food into a form of sugar that is not absorbed in the stomach before being decomposed into glucose due to the actions of several enzymes such as maltase, sucrase, lactase, etc. in the small intestine and absorbed in the body. Therefore, it is expected to be very useful for those who need to control the ingestion of carbohydrates due to obesity, or those who need to regulate blood sugar due to the risk of hyperglycemia or diabetes.

### DESCRIPTION OF DRAWINGS

FIG. 1 is a result of testing the effect of catalase for hydrogen peroxide on a glucose oxidase reaction according to an embodiment of the present invention (w/o catalase: catalase-free control, and w/catalase: catalase-added experimental group).

FIGS. 2 to 7 are graphs showing animal test results according to an embodiment of the present invention:

FIG. 2 is a graph showing a result of measuring blood sugar over time after the enzyme composition of the present invention is orally administered to a rat at 2, 5 or 50 mg per kg of body weight of the rat, and after 30 minutes, glucose is orally administered to the rat at 2 g per kg of body weight of the rat. Units of the y-axis: mg/dL;

FIG. 3 is a graph showing a result of measuring blood sugar over time after the enzyme composition of the present invention is orally administered to a rat at 2, 5 or 50 mg per kg of body weight of the rat, and after 30 minutes, sucrose is orally administered to the rat at 2 g per kg of body weight of the rat. Units of the y-axis: mg/dL;

FIG. 4 is a graph showing a result illustrating an increase in blood sugar per minute for first 30 minutes after the enzyme composition of the present invention is orally administered to a rat at 2, 5 or 50 mg per kg of body weight of the rat, and after 30 minutes, glucose is orally administered to the rat at 2 g per kg of body weight of the rat. Units of the y-axis: mg/dL*min;

FIG. 5 is a graph showing a result illustrating an increase in blood sugar per minute for first 30 minutes after the enzyme composition of the present invention is orally administered to a rat at 2, 5 or 50 mg per kg of body weight of the rat, and after 30 minutes, sucrose is orally administered to the rat at 2 g per kg of body weight of the rat. Units of the y-axis: mg/dL*min;

FIG. 6 is a graph showing a result illustrating an area of the change in blood sugar for 2 hours after the enzyme composition of the present invention is orally administered to a rat at 2, 5 or 50 mg per kg of body weight of the rat, and after 30 minutes, glucose is orally administered to the rat at 2 g per kg of body weight of the rat. Units of the y-axis: mg*min/dL; and

FIG. 7 is a graph showing a result illustrating an area of the change in blood sugar for 2 hours after the enzyme composition of the present invention is orally administered to a rat at 2, 5 or 50 mg per kg of body weight of the rat, and after 30 minutes, sucrose is orally administered to the rat at 2 g per kg of body weight of the rat. Units of the y-axis: mg*min/dL.

### MODES OF THE INVENTION

An enzyme composition of the present invention includes one or more enzymes selected from the group consisting of glucoamylase, sucrase and lactase; glucose oxidase; and transglucosidase.

The glucoamylase, sucrase and lactase, which are enzymes converting carbohydrates such as polysaccharides or disaccharides into glucose, may previously convert carbohydrates into glucose before reaching the small intestine following food ingestion, and convert the converted glucose into gluconolactone due to the action of the glucose oxidase or convert the converted glucose into the form that has a large molecular weight and is not acted on by an enzyme such as maltase, sucrase or lactase due to the action of the transglucosidase, such that glucose may not be used in the body. The enzyme composition of the present invention may regulate sugar metabolism according to such a principle.

According to the present invention, it is effective to further include catalase in addition to the above enzymes. By the action of the glucose oxidase, hydrogen peroxide is generated, and the hydrogen peroxide may inhibit the activity of each enzyme and is not good for the human body. As the catalase eliminates such hydrogen peroxide, a factor that interferes with each enzyme activity of the enzyme composition of the present invention may be excluded, and the composition may be more safely applied to the human body.

Preferably, the enzyme composition of the present invention includes all of the glucoamylase, sucrase, lactase, glucose oxidase, transglucosidase and catalase. In this case, the enzyme composition of the present invention may more effectively convert various forms of carbohydrates contained in food into glucose, and more effectively convert the converted glucose into a form that cannot be used in the body.

In addition, according to the present invention, it is effective to further include one or more types of amylases selected from α-amylase and β-amylase in addition to the enzymes. Accordingly, since the decomposition of polysaccharides, except disaccharides, may be more effectively performed, carbohydrates contained in food may be more effectively converted into a form that cannot be used in the body before the food reaches the small intestine.

Each enzyme is preferably an enzyme that can be used in food, and for example, the enzymes may be obtained by culturing natural microorganisms such as Aspergillus niger, Aspergillus oryzae, and Saccharomyces cerevisiae, or commercially available.

Particularly, these enzymes produced from natural strains of fungi or yeasts such as Aspergillus niger, Aspergillus oryzae, and Saccharomyces cerevisiae usually have an optimal working pH (pH 2 to 7) in acidic and weak acidic conditions, and thus may rapidly work well in the stomach after the ingestion of food (carbohydrates).

To sufficiently exhibit the above action effect of the enzyme composition of the present invention, based on one intake, the glucoamylase may be included to have an activity of preferably 580 U or more, and more preferably 5,800 U or more, the sucrose may be included to have an activity of preferably 20 U or more, and more preferably 200 U or more, the lactase may be included to have an activity of preferably 40 U or more, and more preferably 400 U or more, the transglucosidase may be included to have an activity of preferably 2 U or more, and more preferably 20 U or more, and the glucose oxidase may be included to have an activity of preferably 40 U or more, and more preferably 400 U or more.

When the catalase is included, the main purpose of the catalase is to remove hydrogen peroxide produced by glucose oxidase activity, such that the catalase preferably has an activity 1 time or higher and more preferably 5 times or higher than the activity of the glucose oxidase, and when α-amylase or β-amylase is included, it is included to have an activity of preferably 700 U or more, and more preferably 7,000 U or more.

Since the enzyme composition of the present invention may regulate the absorption of glucose in the body when food is ingested, it may be used for pharmaceuticals, food additives or feed additives to treat, prevent or improve hyperglycemia, diabetes or obesity.

Here, the enzyme composition of the present invention may be formulated according to standards for formulation into a conventional pharmaceutical agent or health supplementary food by the Food and Drug Administration (FDA).

The enzyme composition of the present invention may be diluted by being mixed with a pharmaceutically acceptable carrier or formulated by being encapsulated in a container-shaped carrier depending on an administration method, a dosage form and a treatment purpose by a conventional method.

In addition, the enzyme composition of the present invention may further include a filler, an anticoagulant, a lubricator, a wetting agent, a flavoring agent, an emulsifier or a preservative and thus formulated to prove rapid, sustained or delayed release of an active ingredient after administration to a mammal. In addition, the dose of the present invention may be adjusted according to a patient's condition, an administration route and a dosage form, but the present invention is not limited thereto. While it is obvious to those of ordinary skill in the art that the enzyme composition of the present invention may be used within various ranges, conventionally, in the present invention, it is considered that the enzyme composition can be continuously or intermittently administered daily at an experimentally effective amount, such as 0.1 to 100 mg per kg of body weight.

The enzyme composition of the present invention may be used alone or in combination with a sitologically acceptable carrier as a food additive, and alternatively, in combination with a feed-acceptable carrier as a feed additive, based on the effective amount.

Hereinafter, the present invention will be described in further detail with reference to examples. These examples are merely provided to illustrate the present invention, and it should not be construed that the scope of the present invention is limited by the following examples.

### Example 1. Investigation of Glucose-Reducing Effect According to Combination of Enzymes

1-1. Method

1-1-1. Investigation of Effect of Reducing Maltose-Derived Glucose

10 mL of a 2% (w/v) maltose aqueous solution, as a substrate, was put into each of two prepared 100 mL Erlenmeyer flasks, and heated in a 37° C. constant temperature water bath before use.

To one of the Erlenmeyer flasks, 10 mL of an aqueous solution in which glucoamylase (290,000 U/g) was dissolved alone to have the final concentration of 0.2 mg/mL was added, and to the other flask, 10 mL of an aqueous solution in which glucoamylase (290,000 U/g; derived from Aspergillus niger) and glucose oxidase (20,000 U/g; derived from Aspergillus niger) were dissolved to have the final concentration of 0.2 mg/mL was added. These flasks were well mixed by stirring, reacted in a 37° C. constant temperature water bath for 2 hours, and then boiled on a direct flame for 5 minutes to stop the reaction. In addition, constitutive sugars were analyzed for each reaction sample.

As a control, separately, 10 mL of an aqueous solution in which glucoamylase (290,000 U/g) and glucose oxidase (20,000 U/g) were dissolved to have the final concentration of 0.2 mg/mL was boiled on a direct flame for 5 minutes to inactivate the enzyme, added to 10 mL of a 2% (w/v) maltose aqueous solution, and reacted at 37° C. for 2 hours to analyze constitutive sugars.

The analysis of constitutive sugars was performed using a High Performance Anion-Exchange Chromatography system (HPAEC; ICS-5000, Dionex Co., USA) equipped with an amperometric detector, CarboPac PA-1 (250×4 mm, Dionex Co., USA) was used as a column, and an 18 mM NaOH solution was used as a mobile phase. A flow rate was set to 1.0 mL/min, and a column temperature was set to 25° C.

1-1-2. Investigation of Effect of Reducing Sucrose-Derived Glucose

Investigation was performed by the same method as described in 1-1-1, except that a 2% (w/v) sucrose aqueous solution was used as a substrate, instead of a 2% (w/v) maltose aqueous solution, and sucrase (10,000 U/g; derived from Saccharomyces cerevisiae) was used as an enzyme, instead of glucoamylase (290,000 U/g).

1-1-3. Investigation of Effect of Reducing Lactose-Derived Glucose

Investigation was performed by the same method as described in 1-1-1, except that a 2% (w/v) lactose aqueous solution was used as a substrate, instead of a 2% (w/v) maltose aqueous solution, and lactase (100,000 U/g; derived from Aspergillus niger) was used as an enzyme, instead of glucoamylase (290,000 U/g).

1-1-4. Investigation of Effect of Reducing Glucose

Investigation was performed by the same method as described in 1-1-1, except that a 2% (w/v) glucose aqueous solution was used as a substrate, instead of a 2% (w/v) maltose aqueous solution, and transglucosidase (200 U/g; derived from Aspergillus niger) was used as an enzyme after being dissolved to have the final solution of 2 mg/mL, instead of glucoamylase (290,000 U/g).

In addition, an experimental group using an aqueous solution in which transglucosidase (200 U/g), glucose oxidase (20,000 U/g) and catalase (50,000 U/g; derived from Aspergillus niger) were dissolved to have a final concentration of 2 mg/mL, 0.2 mg/mL and 0.2 mg/mL, respectively, was added.

1-2. Results

Results of the above investigations are shown in Tables 1 to 4, below.

TABLE 1

| Sample | Maltose | Sucrose | Lactose | Glucose | Fructose | Galactose |
|---|---|---|---|---|---|---|
| Maltose (control) | 9.00 ± 0.00 | ND | ND | 0.10 ± 0.00 | ND | ND |
| Maltose/G | 7.25 ± 0.02 | ND | ND | 2.75 ± 0.02 | ND | ND |
| Maltose/G/GO | 8.27 ± 0.02 | ND | ND | 1.73 ± 0.02 | ND | ND |

* G: glucoamylase,
GO: glucose oxidase
* ND: not detected
* Units: g/L

TABLE 2

| Sample | Maltose | Sucrose | Lactose | Glucose | Fructose | Galactose |
|---|---|---|---|---|---|---|
| Sucrose (control) | ND | 7.60 ± 0.41 | ND | ND | ND | ND |
| Sucrose/S | ND | 3.63 ± 0.19 | ND | 3.4 ± 0.10 | 2.53 ± 0.12 | ND |
| Sucrose/S/GO | ND | 2.74 ± 0.10 | ND | 1.76 ± 0.04 | 1.40 ± 0.05 | ND |

* S: sucrase,
GO: glucose oxidase
* ND: not detected
* Units: g/L

TABLE 3

| Sample | Maltose | Sucrose | Lactose | Glucose | Fructose | Galactose |
|---|---|---|---|---|---|---|
| Lactose (control) | ND | ND | 10.40 ± 0.32 | ND | ND | ND |
| Lactose/L | ND | ND | 2.83 ± 0.06 | 4.72 ± 0.14 | ND | 4.77 ± 0.16 |
| Lactose/L/GO | ND | ND | 1.75 ± 0.04 | 2.19 ± 0.06 | ND | 2.38 ± 0.08 |

* L: lactase,
GO: glucose oxidase
* ND: not detected
* Units: g/L

TABLE 4

| Sample | Maltose | Sucrose | Lactose | Glucose | Fructose | Galactose |
|---|---|---|---|---|---|---|
| Glucose (control) | ND | ND | ND | 10.21 ± 0.08 | ND | ND |
| Glucose/TG | ND | ND | ND | 9.52 ± 0.08 | ND | ND |
| Glucose/GO | ND | ND | ND | 9.41 ± 0.05 | ND | ND |
| Glucose/TG/GO | ND | ND | ND | 5.95 ± 0.01 | ND | ND |
| Glucose/TG/GO/C | ND | ND | ND | 4.41 ± 0.04 | ND | ND |

* TG: transglucosidase,
GO: glucose oxidase,
C: catalase
* ND: not detected
* Units: g/L

When glucose oxidase was treated alone as described above, a significant effect of reducing glucose was confirmed, compared to the effect expected when glucoamylase, sucrase, lactase or transglucosidase was treated with glucose oxidase.

That is, as shown in the result of the “glucose/GO” sample in Table 4, when glucose oxidase was treated alone, a glucose-reducing effect of approximately 7.8% was shown, whereas when glucose oxidase was treated with glucoamylase, a glucose-reducing effect of approximately 37% was shown (refer to Table 1), when glucose oxidase was treated with sucrase, a glucose-reducing effect of approximately 48.2% was shown (refer to Table 2), when glucose oxidase was treated with lactase, a glucose-reducing effect of approximately 53.6% was shown (refer to Table 3), and when glucose oxidase was treated with transglucosidase, a glucose-reducing effect of approximately 41.7% was shown (refer to Table 4).

In addition, when glucose oxidase was treated with catalase, a glucose-reducing effect was more increased.

### Example 2. Investigation of Effect of Catalase on Glucose Oxidase Reaction-Derived Hydrogen Peroxide

A substrate solution was prepared by dissolving glucose in 100 mL of a buffer solution containing each of 0.1M sodium citrate (pH 3.0), 0.1M sodium acetate (pH 5.0) and 0.1M sodium phosphate (pH 7.0) to have a concentration of 1% (w/v), 1 mL of glucose oxidase (400 U/mL) and 1 mL of catalase (2,000 U/mL) were added to the substrate solution, and then an amount of hydrogen peroxide was measured at 37° C. over time (0, 1, 2, 4, 8, and 24 hrs) using a hydrogen peroxide quantification kit (350-H₂O₂, ITS, China).

In addition, for comparison, a control excluding catalase was used.

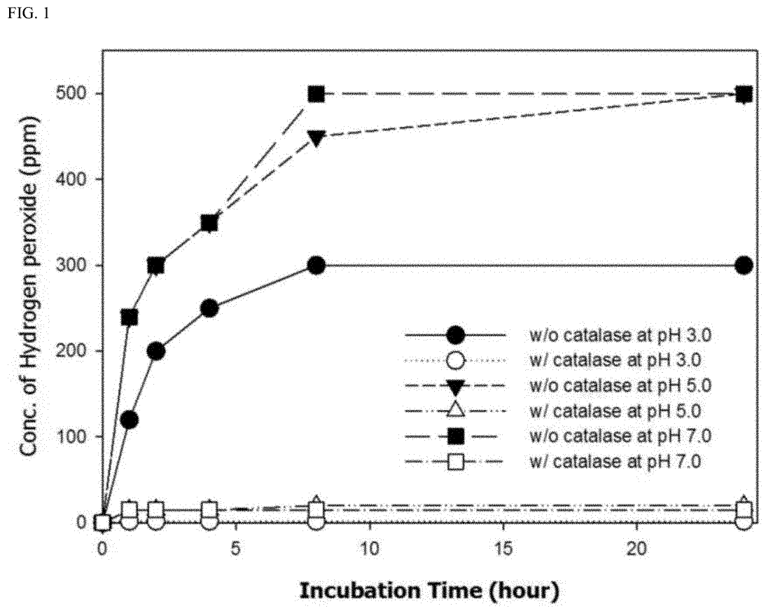

As a result, referring to FIG. 1, hydrogen peroxide generated by a glucose oxidase reaction was effectively removed by catalase.

### Example 3. Animal Experiment

3-1. Method

Enzyme compositions were prepared, as shown in Table 5 below, using transglucosidase, sucrase, lactase, glucoamylase, glucose oxidase, catalase, and additionally α-amylase.

TABLE 5

| | | | Manufacturer/ |
|---|---|---|---|
| | Activity | | Manufacturing |
| Enzyme | (/400 mg) | Origin | country |
| Transglucosidase | 20 U | Aspergillus niger | Nensys Co., Ltd/ |
| | | | Korea |
| Sucrase | 200 U | Saccharomyces | Orchid/China |
| | | cerevisiae | |
| Lactase | 400 U | Aspergillus niger | Adavanced Enzyme |
| | | | Technologies/India |
| α-Amylase | 7,000 U | Aspergillus oryzae | Shandong Longda/ |
| | | | China |
| Glucoamylase | 5,800 U | Aspergillus niger | Shandong Longda/ |
| | | | China |
| Glucose oxidase | 400 U | Aspergillus niger | Nensys Co., Ltd/ |
| | | | Korea |
| | | | Nensys Co., Ltd/ |
| Catalase | 2,000 U | Aspergillus niger | Korea |

The enzyme composition was orally administered to a rat (Sprague Dawley, average body weight: 160 g, 7-week-old) at 2, 5 or 50 mg per kg of the body weight of the rat, and after 30 minutes, glucose or sucrose was orally administered thereto at 2 g per kg of the body weight of the rat, followed by measuring blood sugar over time.

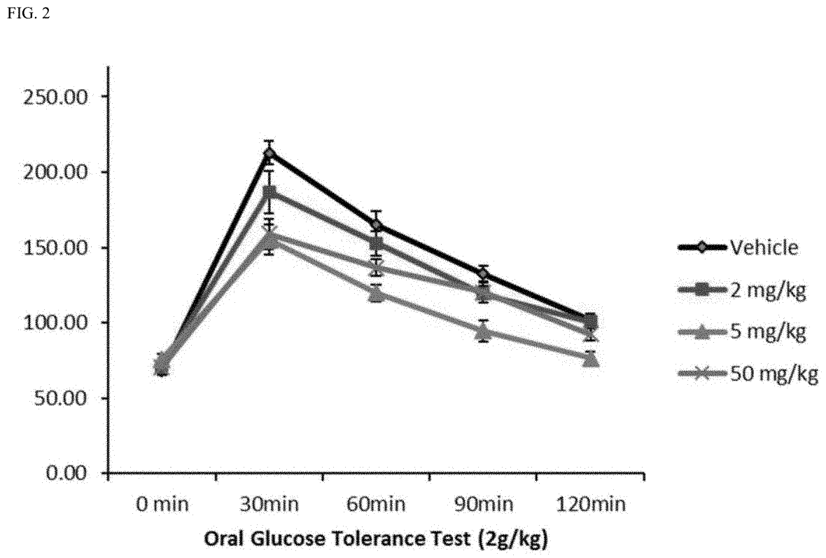

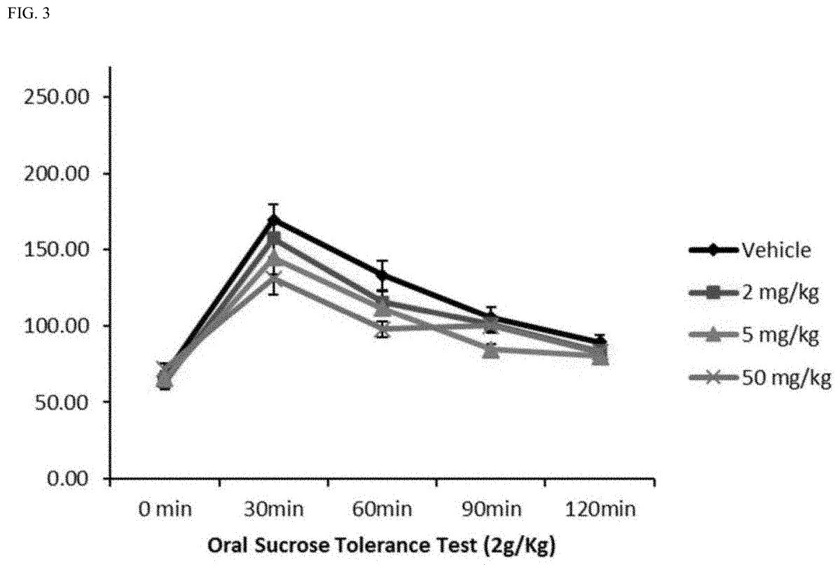

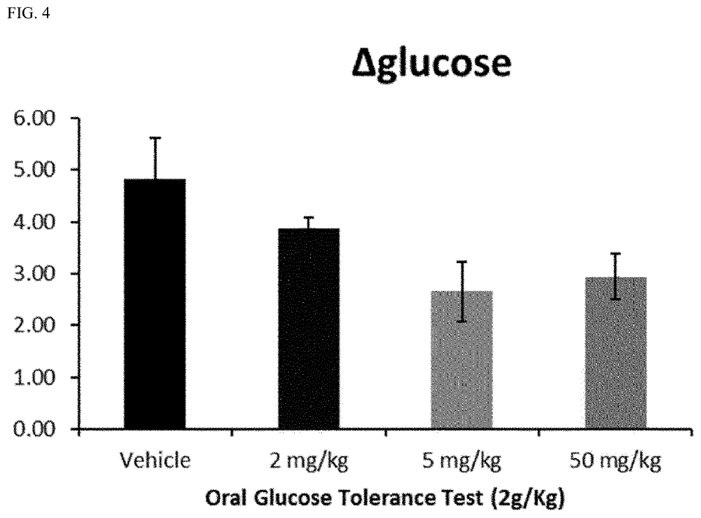

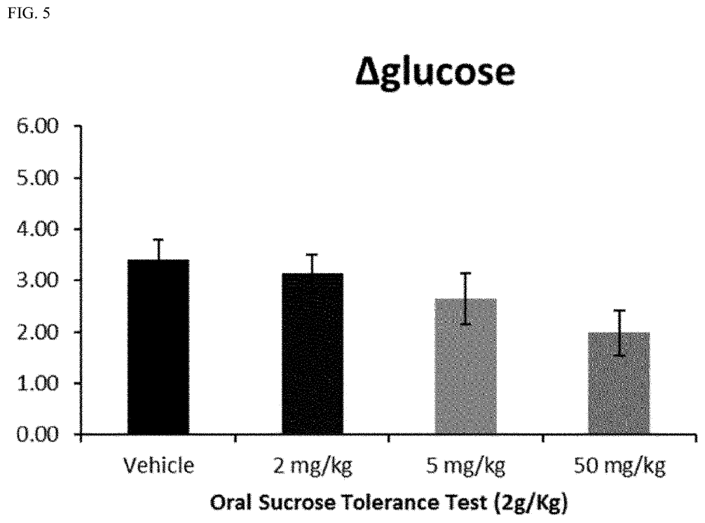

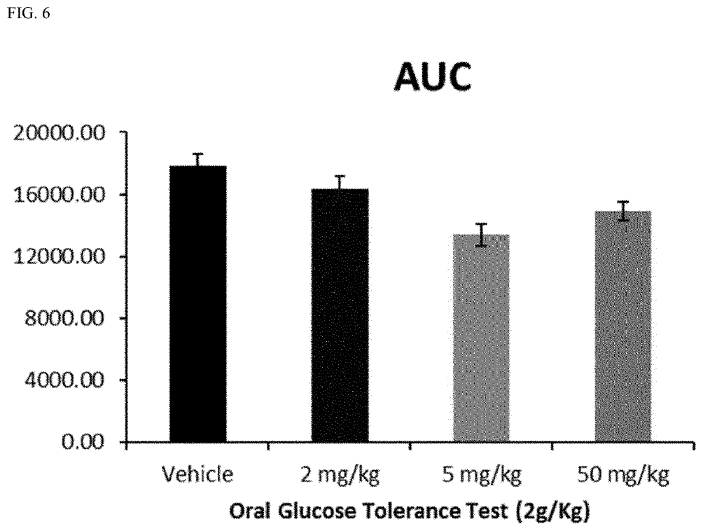

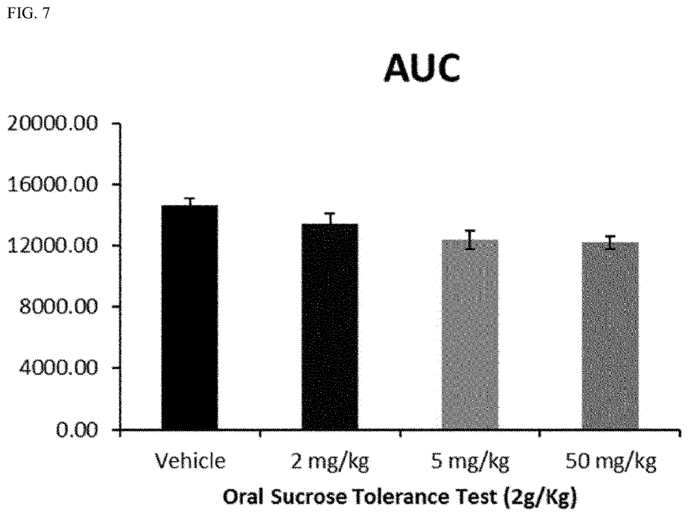

As a result, referring to FIGS. 2 to 7, when the enzyme composition was administered, an increase in blood sugar according to the ingestion of glucose or sucrose was reduced.

[Enzyme Activity Test]

1. Glucoamylase Test

1-1. Definition of Activity

1 U refers to the activity of an enzyme that hydrolyzes 1 mg of starch for 1 hour under conditions of pH 6.0 and 40° C., and U/g is indicated for a solid sample, and U/mL is indicated for a liquid sample.

1-2. Test Procedure

25 mL of a 2% (w/v) soluble starch solution was added to each of two test tubes, 5 mL of a 0.1M citrate buffer solution (pH 6.0) was added thereto, and then the test tube was well stirred and maintained in a 40° C. constant temperature water bath for 5 minutes. 2 mL of the test solution was added to a test tube for a test group and then well stirred for 30 minutes. 0.2 mL of a 20% (w/v) sodium hydroxide solution was added to each of two test tubes for a test group and a blank test group and then well stirred, followed by cooling. 2 mL of a 0.1M acetate buffer solution (pH 6.0) was added to the blank test tube. 5 mL of the test solution was taken from each of the two test tubes, mixed with 10 mL of a 0.1M iodine solution and 15 mL of a 0.1M sodium hydroxide solution and then well stirred, followed by reaction in a dark place for 15 minutes. 2 mL of a 2M sulfate solution was added, and titrated with a 0.05M sodium thiosulfate solution until a blue color disappeared. The activity was calculated according to the following equation.

X=(a−b)c×90.005×32.2/5×½×n×2=579.9×(a−b)c×n

a: Optimal volume (mL) of blank test group

b: Optimal volume (mL) of test group

c: Concentration (mol/L) of standard sodium hyposulfite solution

90.05: Weight of glucose (in 1 mL of standard sodium hyposulfite solution)

32.2: Total volume (mL) of reaction solution

5: Volume (mL) of absorbed reaction solution

½: Absorbed enzyme solution 2 mL=1 mL

n: Expansion rate

2: Conversion of reaction time of 30 minutes into 1 hour

2. Sucrase Test Method

2-1. Definition of Activity

In the definition of activity in this test method, when a test was performed according to the following test method, an amount (g) of enzyme required for converting 1 mg of sucrose into glucose and fructose for 5 minutes is determined as one unit.

2-2. Test Procedure

5 mL of a 6.5% (w/v) sucrose solution (substrate solution) was put into a test tube (set as a blank test group or a test group per sample) and heated in a 20° C. constant temperature water bath. At this time, 10 mL of the prepared test solution was heated at the same temperature. 1 mL of the test solution (0.5 U/mL) was added to each test tube containing the substrate solution and vigorously stirred. A blank test group for the test solution was prepared by immersing the test solution in boiling water for 10 minutes, and then cooling on ice for 5 minutes and adding 1 mL of an inactivated enzyme solution.

For glucose standard preparation, 1 mL of a 0.3% (w/v) glucose solution was added to three test tubes containing 5 mL of a 6.5% (w/v) sucrose solution (substrate solution).

To prepare a blank test group for a substrate, 1 mL of distilled water was added to three test tubes containing 5 mL of a 6.5% (w/v) sucrose solution (substrate solution). After exactly 30 minutes, 7 mL of a 3,5-DNS solution¹⁾ was added and stirred to stop an enzyme reaction. Likewise, 7 mL of a 3,5-DNS solution²⁾ was added to each of 3 mL of a 0.3% (w/v) glucose solution, 3 mL of a 6.5% (w/v) sucrose solution (substrate solution), and 3 mL of an inactivated enzyme reaction solution and then vigorously mixed.

All test tubes were heated in hot water for 10 minutes, and cooled on ice for 5 minutes. 40 mL of distilled water was added to each test tube, and vigorously mixed.

After being maintained at room temperature for 10 minutes, and the absorbance of the test solution was measured at 515 nm with the absorbance of distilled water set to “0.”

Sucrase activity (μ/g) of test solution=[(A_{U}−A_{B})/(A_{S}−A_{W})]×(0.5/C)

A_{U}: Average absorbance of test solution

A_{B}: Average absorbance of blank test group for test solution

A_{S}: Average absorbance of glucose standard group

A_{W}: Average absorbance of blank test group for substrate

C: Concentration (g/mL) of test solution

0.5: (3 mg glucose*5 min unit definition)/30 min reaction

1) 3,5-DNS solution: 308 g of sodium potassium tartrate tetrahydrate and 19.4 g of sodium hydroxide were put into a 1000 mL flask, and dissolved in DW. 10.7 g of 3,5-DNS acid was added and dissolved. 8.33 g of phenol was transferred to a third container, 1.83 g of sodium hydroxide and 8.33 g of sodium metabisulfite were dissolved therein. The resulting solution which was prepared was used within 48 hours, and filtered using a glass fiber. 3 mL of a 0.3% (w/v) glucose solution was added to 200 mL of the solution.

3. Lactase Test Method

3-1. Definition of Activity

One unit (U) refers to the activity of an enzyme that decomposes 1 μmol of a substrate for 1 minute under the above test conditions.

3-2. Test Procedure

A test tube which has a diameter of 25 mm and a length of 150 mm was prepared. 2 mL of a substrate solution (o-nitrophenyl-β-D-glactopyranoside, 7.4 mg/mL, pH 4.5) was maintained at 37° C. for 10 minutes. A test solution was put into the test tube, and an equal amount of distilled water was added to a blank test group, followed by a reaction for 15 minutes. 2.5 mL of a 10% (w/v) sodium carbonate solution was added and stirred to stop the reaction, and 20 mL of distilled water was added to dilute the above-prepared solution to 25 mL and then vigorously stirred. Absorbance was measured at 420 nm.

A standard curve was plotted as follows. 139 mg of o-nitrophenol was put into a 500 mL flask, and dissolved in 10 mL of 95% ethanol, thereby preparing a 2 mM o-nitrophenol solution. This solution was diluted to various concentrations in a 1% (w/v) sodium carbonate solution.

TABLE 6

| | Mixed with 1% (w/v) sodium | |
|---|---|---|
| 2 mM o-nitrophenol | carbonate solution to adjust a | Concentration of |
| solution (mL) | volume to 100 ml | o-nitrophenol |
| 5.0 | | 0.10 |
| 7.0 | | 0.14 |
| 9.0 | | 0.18 |

* set to R² > 0.99

The activity of lactase was calculated according to the following equation.

Lactase activity of test solution (U/g)=[(As−B)(25)]/[(ε)(15)(W)]

As: Absorbance of test group

B: Absorbance of blank test group

E: Standard absorbance of o-nitrophenol per μmol

25: Final volume

15: Reaction time (min)

W: Weight (g) of sample added to first test solution (1 mL)

4. Glucose Oxidase Test Method

4-1. Definition of Activity

1 U of glucose oxidase refers to the activity of the enzyme that oxidizes 1 μmol of β-D-glucose to D-gluconic acid under conditions of 37° C. and pH 6.0 for 1 minute.

4-2. Test Procedure

A standard substance was prepared by further diluting the diluted glucose oxidase (HRP, GenView DH165-2, 4 U/mL) with a 0.1M phosphate buffer solution (pH 6.0) according to the following Table 7.

TABLE 7
Standard dilution factor

| | | Initial |
|---|---|---|
| Standard | Initial | volume -> Final |
| solution | concentration -> Final | volume |
| No. | concentration (U/ml) | (mL) |
| 1 | 4 -> 0.8 | 0.2 -> 1 |
| 2 | 4 -> 1.2 | 0.3 -> 1 |
| 3 | 4 -> 1.6 | 0.4 -> 1 |
| 4 | 4 -> 2.0 | 0.5 -> 1 |
| 5 | 4 -> 2.4 | 0.6 -> 1 |
| 6 | 4 -> 2.8 | 0.7 -> 1 |

The standard substance was manipulated according to Table 8 and then maintained at 37° C. for 5 minutes without glucose oxidase. 0.1 mL of a glucose oxidase solution was added to perform a reaction for 3 minutes. 2 mL of 2M H₂SO₄ was added and mixed by stirring, and then put into a 1-cm cell to measure absorbance at 540 nm, followed by plotting a standard curve. (Y-axis: concentration of glucose oxidase/X-axis: set as OD value/R2>0.995)

TABLE 8
Sample-added weight

| | Blank test | Test solution |
|---|---|---|
| Item | tube (ml) | test tube (ml) |
| 1% (w/v) dianisidine solution | 2.6 | 2.5 |
| 1.8% (w/v) glucose solution | 0.3 | 0.3 |
| Peroxidase (100 U/mL) solution | 0.1 | 0.1 |
| Test solution | — | 0.1 |

A test solution was subjected to testing performed according to Table 8 to measure absorbances of a blank test solution and the test solution, and then glucose oxidase activity of the test solution was calculated according to the following equation.

Y=K(AE−AB)×D

Y: Glucose oxidase activity (U/mL) of test solution

AE: OD value of test solution

AB: OD value of blank test solution

K: Slope on standard curve

D: Dilution factor

5. Transglucosidase Test Method

5-1. Definition of Activity

When testing was performed according to test conditions of this test method, an amount of enzyme that generates 1 μg of 4-nitrophenol from 4-nitrophenyl α-D-glucopyranoside for one minute is referred to one unit.

5-2. Test Procedure

1 mL of 10 mM p-NPG, 0.5 mL of 0.2M sodium acetate buffer (pH 5.5) and 100 μL of a 5% (w/v) pyridine solution were added to a sealable test tube, and pre-heated in a 50° C. constant temperature water bath for 5 minutes. After 5 minutes, 200 μL of a test solution was added, stirred, and reacted at 50° C. for 10 minutes. After exactly 10 minutes, a reaction solution was taken from the constant temperature water bath, and 200 μL of 0.2M sodium carbonate was added to stop an enzyme reaction. After standing at 25° C. for 10 minutes, an absorbance (A₄₂₀) value was measured at 420 nm using a spectrophotometer (T). Here, as a blank test group, 1 mL of 10 mM p-NPG, 0.5 mL of 0.2 M sodium acetate buffer (pH 5.5) and 100 μL of a 5% (w/v) pyridine solution were added, mixed and then maintained in a 50° C. constant temperature water bath for 10 minutes, 200 μL of 0.2M sodium carbonate was added and mixed, and then 200 μL of the test solution was added again. Absorbance (A₄₂₀) of the blank test group was measured (B).

A p-nitrophenol standard curve was plotted as follows, and a slope (a) was obtained. 1,780 μL, 1,760 μL, 1,740 μL, 1,720 μL and 1,700 μL of distilled water were added to test tubes containing 20 μL, 40 μL, 60 μL, 80 μL and 100 μL of 0.1 mg/mL p-nitrophenol, respectively, and 200 μL of 0.2 M sodium carbonate was added to the respective test tubes, followed by stirring well. The final concentrations of p-nitrophenol were 1 μg/mL (C₁), 2 μg/mL (C₂), 3 μg/mL (C₃), 4 μg/mL (C₄) and 5 μg/mL (C₅), respectively. Absorbances (A₁, A₂, A₃, A₄, and A₅) for the resulting substances were measured at 420 nm. Regression analysis is performed with the concentrations (C₁, C₂, C₃, C₄, and C₅) set to the x-axis, and the absorbances (A₁, A₂, A₃, A₄, and A₅) set to the y-axis, and a slope a is obtained.

Transglucosidase activity of test solution (U/g)=1/W×(T−B)/a× 1/10×2

W: Amount (g) of sample added to enzyme reaction

T: Absorbance (A₄₂₀) of enzyme reaction solution

B: Absorbance (A₄₂₀) of blank test solution

a: Slope of standard curve

1/10: Reaction time correction factor

2: Volume correction factor

6. Catalase Test Method

6-1. Definition of Activity

One unit of catalase in this test method refers to the activity of catalase that decomposes 1 μmol of hydrogen peroxide under conditions of pH 6.8 and 30° C. for one minute.

6-2. Test Procedure

5 mL of a 0.075% (v/v) hydrogen peroxide solution (pH 6.8) was transferred to a flask, and maintained in a 30° C. constant temperature water bath for 5 minutes. Here, 1 mL of a test solution (pH 6.8) was added, and reacted for 5 minutes. Afterward, 2 mL of 1N H₂SO₄ was added to stop the reaction. 1 mL of a 10% (w/v) potassium iodide solution and one drop of 1% (w/v) ammonium molybdate, and two or three drops of an indicator [1% (w/v) starch-iodide] were added. Free iodine was titrated using 0.01N sodium thiosulfate, and the amount used is set as V1. A blank test group was prepared by adding 2 mL of 1N H₂SO₄ and 1 mL of the test solution (pH 6.8) but excluding a substrate solution, and a suitable amount of the blank test group was set as V2. Activity was calculated according to the following equation.

Catalase activity of test solution (U/g)=(V2−V1)×(0.01/2)×1000×DT/5

DT: Dilution factor

V2: Amount (mL) of sodium thiosulfate used in titration of blank test group

V1: Amount (mL) of sodium thiosulfate used in titration of test group

0.01: Concentration (mol/L) of thiosulfate solution

7. A-Amylase Test Method

7-1. Definition of Activity

One unit (U) of α-amylase refers to an amount of enzyme that dextrinizes soluble starch at a rate of 1 g per hour in the presence of a sufficient amount of β-amylase at 30° C.

7-2. Test Procedure

Twenty 13×100 mm test tubes were set as one set, and then 5 mL of an iodine reagent¹) was put into each test tube and maintained in a constant temperature water bath at 30±0.1° C. 20 mL of a substrate solution previously treated in a water bath for 20 minutes was put into a 50 mL Erlenmeyer flask, 5 mL of 0.5% sodium chloride solution previously treated for 20 minutes in the same water bath was added to 2% (w/v) soluble starch (pH 4.8) maintained at a constant temperature, and then the flask was immediately sealed, stirred and then maintained in the water bath. 5 mL of the test solution was added at a test starting time and maintained in a water bath. After 10 minutes, 1 mL of the reaction mixed solution in the 50 mL Erlenmeyer flask was added to the test tube containing the iodine reagent and well stirred, followed by immediately comparing the content with a standard color²⁾ obtained from a colorimeter. Behind a colorimeter plate, tubes containing water were used. Repeated comparative experiments were carried out by the same method at regular and accurate time intervals and continued until the same color as the standard color was obtained. Every hour at which the test solution was obtained was recorded. The activity of the test solution was obtained according to following equation.

Amylase activity (solution) of test solution=24/(W×T)

W: Amount (g) of enzyme contained in 5 mL of test solution

T: Dextrinization time (min)

24: Calculated value with starch substrate weight (0.4 g) for 60 minutes

1) Iodine reagent: 20 g of potassium iodide was dissolved in 300 mL of water, 2.0 mL of an iodine stock solution (5.5 g of iodide and 11.0 g of potassium iodide were dissolved in purified water to adjust a final volume to 250 mL) was added thereto, and then water was added to have a final volume of 500 mL.

2) Standard color: 25 g of cobalt chloride (CoCl₂.6H₅O) and 3.84 g of potassium dichromate were dissolved in 0.01N hydrochloric acid to have a final volume of 100 mL.

1. An enzyme composition for regulating sugar metabolism, comprising:
one or more enzymes selected from the group consisting of glucoamylase, sucrase and lactase;
glucose oxidase; and
transglucosidase.

2. The composition of claim 1, further comprising catalase.

3. The composition of claim 2, wherein all of glucoamylase, sucrase, lactase, glucose oxidase, transglucosidase and catalase are comprised.

4. The composition of claim 3, further comprising one or more types of amylases selected from α-amylase and β-amylase.
